(19) United States
(12) Patent Application Publication
(10) Pub. No.: US 2009/0105206 A1
(43) Pub. Date: Apr. 23, 2009

(54) Copper Melphalan And Copper Tegafur As Anti-Tumor Agents

(76) Inventor: Gust Ronald, Teltow (DE)

Correspondence Address: MORRIS MANNING MARTIN LLP, 3343 PEACHTREE ROAD, NE, 1600 ATLANTA FINANCIAL CENTER, ATLANTA, GA, 30326, US

(21) Appl. No.: 11/791,394

(22) PCT Filed: Nov. 24, 2005
(86) PCT No.: PCT/EP2005/012590

§ 371 Date: May 23, 2007

(30) Foreign Application Priority Data
Nov. 24, 2004 (DE) 10 2004 056 727.1

(51) Int. Cl. A61K 31/555; A61K 31/30; A61P 35/04

(52) U.S. Cl. 514/184; 514/499

(57) ABSTRACT

The present invention relates to the use of copper compounds of general formula Cu(L)₂ or hydrates thereof, wherein the ligands L independently denote melphalan and tegafur, as anti-tumor agents.

The present invention relates to the use of copper compounds of general formula Cu(L)₂ or hydrates thereof, wherein the ligands L independently denote melphalan and tegafur, as anti-tumor agents.

The development of new chemotherapeutics that are highly effective in treating malignant tumors and also possess high selectivity, and therefore entail as few side effects as possible, is an important aim in current research. A large number of cytostatics are already known which, with the aid of their different mechanisms of action, may be divided into several groups.

For example pyrimidine, purine and pteridine derivatives, such as fluoro- or bromouracil, are anti-metabolites which, owing to their property of being structurally similar to essential natural metabolites and of being able to replace these in the organism or being able to compete therewith, can inhibit or misroute the progress of biological processes.

The group of mitotic inhibitors includes for example the plant ingredients colcemide, podophyllin derivatives and vinka alkaloids which affect the progress of mitosis in cells, whereby cell division is prevented and fast-growing tumor cells are thus killed.

A further group of cytostatics are alkylating compounds, of which the effect is primarily based on the alkylation of nucleic acids, whereby the DNA is changed and as a consequence thereof cell division is impaired and ultimately cell death is initiated. Examples of alkylating cytostatics are N-lost derivatives such as chloroambucil, cyclophosphamide and melphalan (4-[bis-(2-chloroethyl)-amino]-L-phenylalanine).

By way of example melphalan was proposed for the treatment of various cancers. Alkeran®, which contains melphalan as the active ingredient, is a pharmaceutical composition for use with multiple myeloma, ovarian carcinoma and mamma carcinoma. Studies on melphalan in the case of advanced prostate cancer have also been carried out (Canada, A., et al, Cancer Chemother. Pharmacol. 1993, 32(1), 73-7; Houghton, A., et al, Cancer Treat. Rep. 1977, 61(8), 923-4). Further prior art which relates to the uses of melphalan are for example Stolfi, R. L. et al, J. Natl. Cancer Inst. 1988 80(/1), 52-5; Fisher, B. et al, N. Engl. J. Med., 1975, 292(16), 117-122) (Breast Cancer); Cornwell G. G. et al, Cancer Treat. Rep., 1982, 66(3), 475-81; Gola, A. et al, Folia Haematologica, 1990, 117, 167 (myeloma/leukemia); Hendriks, J. M. et al, Ann. Thorac. Surg., 1988, 66(5), 1719-25; Steven, M. P. et al, Gynecologic Oncology, 1986, 23, 168 (adenocarcinoma); Vrouenraets, B. C. et al, J. Surg. Oncol., 1997, 65(2), 88-94; Haffner, A. C. et al, Br. J. Dermatol., 199, 141 (Suppl Nov.) 935-36 (sarcoma) and Fraker D. L. et al, J. Clin. Oncol., 1996, 14(2), 479-489; Ferdy, L. et al, Annals of the New York Academy of Sciences, 1993, 680, 391-400 (melanoma)).

R. Tobey et al, Cancer Research 45 (1985) describe temporally separate applications of a metal-containing solution and a melphalan-containing solution. The resistance of cells to melphalan was investigated in this case.

In general the fact that many of the known anti-tumor active ingredients are very toxic and furthermore act as carcinogens or mutagens themselves is a serious problem when using cytostatics. Moreover the active ingredients often only have very restricted use since effective cancer treatment is dependent on the type of malignant tumor being treated and the form, progression and stage of the respective disease. Hence it has been found in the case of melphalan that it has only limited efficiency and breadth of application for anti-tumor treatment.

WO03/004014 discloses an anti-tumor agent that is improved compared with melphalan. Therein compounds of formula D₂-M-T are described which comprise a metal atom, such as copper or manganese, two β diketon ligands and one ligand which is a substance with at least one N-, O- or S-containing group, for example melphalan. In in vivo tests these metal compounds have proven to be effective in treating, for example, adenocarcinoma, sarcoma, leukemia, melanoma and kidney cell carcinoma, it having been possible to demonstrate superior effectiveness compared with melphalan.

In view of the large number of people who are currently suffering from a wide variety of tumor diseases, there is still a continuing need for new cytostatics with high effectiveness and the fewest possible side effects, despite the great diversity of compounds with anti-tumor effectiveness that have been previously described.

The object underlying the present invention was therefore to provide new approaches to effective anti-tumor treatment.

This object is achieved according to the invention in that within the scope of the present invention it has surprisingly been found that compounds of general formula

Cu(L)₂.(H₂O)ₓ (I)

wherein L is in each case independently selected from melphalan and tegafur and x=0, 1 or 2, have high anti-tumor effectiveness and are therefore suitable for use as an active ingredient in treating malignant tumors.

The present invention therefore relates to the use of one of more compounds of formula (I) in anti-tumor treatment.

The compound of formula (I) optionally contains crystallization water, in particular two molecules of H₂O (x=2).

The ligands L coordinated with the copper atom are 4-[bis(2-chloroethyl)amino]-L-phenylalanine) (melphalan) or/and 5-fluoro-1-(tetrahydro-2-furyl)-uracil (tegafur). The two ligands L are preferably melphalan or tegafur, they are particularly preferably both L melphalan ligands.

The particularly preferred compound according to the invention is Cu(melphalan)₂ or the corresponding crystallization water-containing compound. This copper compound is basically known (M. D. Joesten et al, Inorganica Chimica Acta, 159 (1989) 143-148), wherein it has been proposed that the melphalan molecules act as two-dentate ligands which together with two molecules of water arrange themselves around the Cu atom in a tetragonalbipyramidal manner. The melphalan groups are present in the four equatorial positions around the copper; the water molecules are axially arranged relative to the copper atom. Joesten et al demonstrate a possibility for synthesis for compounds according to the invention. Possible anti-tumoral activity of the compounds is not addressed or even investigated. The astonishing anti-tumor effects of Cu(melphalan)₂ were first established within the scope of the present invention.

The compounds of formula (I) proved to be very effective anti-tumor agents. The compounds according to the invention are particularly suitable for treating cancer of the colon, brain tumors, eye tumors, pancreatic carcinomas, bladder carcinomas, lung cancer, breast cancer, ovarian tumors, cancer of the uterus, bone tumors, gall bladder and bile duct carcinomas, head-neck tumors, skin cancer, testicular cancer, kidney tumors, germ cell tumors, liver cancer, leukemia, malignant lymphoma, nerve tumors, neuroblastomas, prostate cancer, soft tissue tumors, esophageal cancer and carcinomas in the case of unknown primary tumors.

Investigations have shown that the compounds of formula (I) and in particular Cu(melphalan)₂ primarily allow effective treatment of kidney and lung cancers. In the process it has been found that the Cu compounds of formula (I) used according to the invention have a much higher anti-tumor effectiveness than the ligands themselves, i.e. melphalan and tegafur. This increase in effectiveness achieved in the case of treatment with the copper compounds according to formula (I) can be attributed to a synergistic effect which is obtained by the incorporation of the melphalan and tegafur ligands into a Cu compound according to the present invention.

In addition to their high effectiveness in combating tumors, it has also been found that the compounds of formula (I) have immunomodulatory and anti-proliferative properties as well as an anti-angiogenetic effect. Compared with conventional anti-tumor agents the compounds according to the invention also have greatly increased hydrolysis stability, whereby they may be used in broad sectors of tumor treatment. Furthermore it has been found that the compounds of formula (I) selectively damage tumor tissue and substantially do not affect healthy tissue. The compounds of formula (I), in contrast to most of the known chemotherapeutics, therefore allow anti-cancer treatment without side effects or with only minor side effects.

A further advantage of the copper compounds used according to the invention is that these compounds do not induce resistance to a pharmaceutical composition and under certain circumstances are capable of bringing about apoptosis of cancer cells.

The particularly preferred copper compounds according to the invention Cu(melphalan)₂ is a blue solid which is chemically stable and has a long-lasting effect. This compound can moreover overcome the blood-brain barrier, and this therefore allows treatment of brain tumors. This compound has proven to be much more effective than melphalan, as could be demonstrated for example by investigations of kidney cell carcinoma and lung metastases formation. Comparable effectiveness could be demonstrated for the additional preferred compound according to the invention: Cu(tegafur)₂. In contrast to tegafur on its own, which has only a short period of effect and causes side effects such as diarrhea and stomatitis and therefore offers only limited application possibilities, the copper-tegafur compound according to the invention has proven to be an anti-tumor agent that does not exhibit these drawbacks and is also much more effective.

According to the present invention, compounds of formula (I) may be used individually or as mixtures of two or more of these compounds for anti-tumor treatment. The compounds of formula (I) can optionally be formulated or administered together with the conventionally used pharmaceutical additives and auxiliaries known to a person skilled in the art. Examples of such additives or auxiliaries are physiologically acceptable carrier substances, diluents, colorings or/and flavorings.

According to the invention the copper compound of formula (I) can be in a form suitable for topical, parenteral, intravenous, intramuscular, subcutaneous, intraperitoneal or transdermal administration. The copper compound of formula (I) is preferably in the form of tablets or provided as intravenous injection or infusion. In certain cases the compounds of formula (I) can be injected in a targeted manner into body cavities or via a catheter into the blood vessels of the tumor regions or the organ in which the tumor is situated.

The compounds of formula (I) or mixtures of such compounds may also be administered together with other active ingredients, wherein these other active ingredients may, for example, also be anti-tumor agents, antibiotics or substances with other effects.

According to the present invention the compound of formula (I) can be used in various stages of the tumor disease to be treated. Thus the copper compound used according to the invention can alleviate the symptoms associated with the tumor disease, reduce the extent of the tumor disease (for example a reduction of tumor growth), stabilize the state of the tumor disease (for example an inhibition of the tumor growth), prevent further spreading of the tumor disease (for example metatastic spread), prevent an occurrence or reoccurrence of a tumor disease, and delay or slow down the progression of the tumor disease (for example a reduction in tumor size). The compound of formula I is administered in a quantity that is sufficient to achieve the desired aim in each case. The respective effective quantity depends on various factors, such as the choice of complex, the manner of administration, the type and extent of the tumor disease and the age, weight and general condition of the patient. The compounds of formula (I) are preferably administered in a dose of 1 μg/kg body weight of the patient up to 8 mg/kg body weight of the patient, preferably up to 7.5 mg/kg body weight of the patient, even more preferably up to 5 mg/kg body weight of the patient. In particular 1 μg/kg body weight of the patient to 0.5 mg/kg body weight of the patient and particularly preferably 10 μg/kg body weight of the patient to 0.1 mg/kg body weight of the patient of the compounds according to the invention are administered.

The copper compounds of formula (I) may be easily prepared according to the synthesis instructions described in the prior art (see for example M. D. Joesten, Inorganica Chimica Acta, 159 (1989) 143-148). A further important advantage of the present invention is that the copper compounds used according to the invention are easily synthetically accessible with a high level of purity, and this represents an important prerequisite for the provision thereof as a pharmaceutical composition.

The present invention also relates to the use of one or more compound(s) of formula (I) for preparing a drug for treating tumors.

The present invention will be described in more detail by the following figures, examples and test results.

### FIGURES

FIG. 1 shows an IR spectrum (KBr) of Cu(melphalan)₂.

FIG. 2 shows a mass spectrum of Cu(melphalan)₂.

FIG. 3 is the representation of a proposed structure for Cu(mel)₂.2H₂O. The molecular composition and the molecular weight are stated under the structural formula.

FIG. 4 shows a synthesis model of the synthesis of Cu(Tf)₂ according to example 2.

FIG. 5 is graph showing a comparison of kidney weight and kidney volume of control mice treated according to example 3 and mice treated with Cu(mel)₂ according to the present invention.

FIG. 6 is a graph showing the lung weight and the number of lung metastases in mice treated according to example 3. The control group (solvent), Cu(mel)₂ group (5 mg/kg/D 10-18), Cu(mel)₂ group (7.5 mg/kg/D 10-18) and the mice treated with melphalan only are compared.

FIG. 7 shows a comparison of the vessel densities of the control mice treated according to example 3 and mice treated with Cu(melphalan)₂ (CD 31 evaluation).

FIG. 8 shows the mean tumor volume during the course of treatment with Cu(melphalan)₂ compared with melphalan.

FIG. 9 shows the relative tumor volumes (RTV) for groups treated with copper melphalan or with melphalan alone.

FIG. 10 shows a statistical analysis of the two dose groups from FIG. 9.

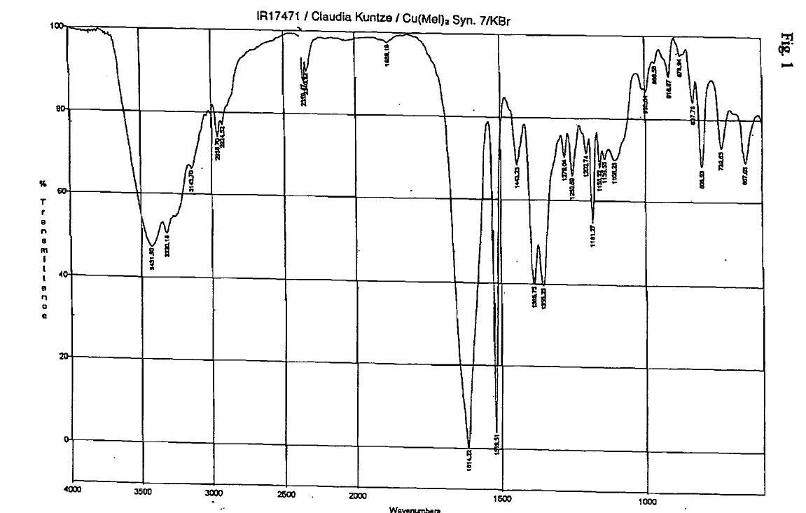

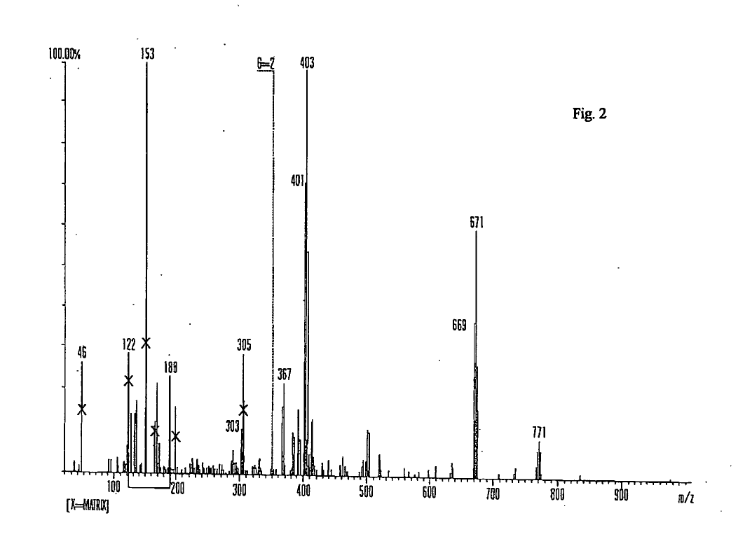

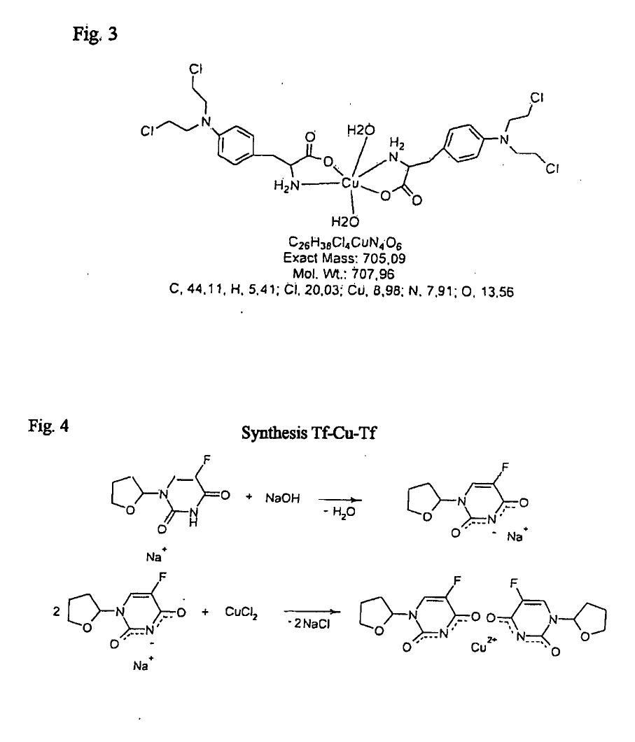

### EXAMPLES

### 1. Synthesis of Cu(melphalan)₂

The synthesis was carried out according to Joesten, M. D., Inorganica Chimica Acta, 159 (1989) 143-148.

0.30 g melphalan (0.98 mmol) were added while stirring to a suspension of Cu(OH)₂ (0.050 g, 0.52 mmol in 3.5 ml water). After 1 hour a blue-purple precipitate formed which was filtered under vacuum and washed with a large quantity of hot water.

### 2. Synthesis of Cu(Tegafur)₂

0.20027 g tegafur were added to 10 ml of a 0.1 N ethanolic NaOH and heated under reflux to 62 to 65° C., wherein everything dissolved. 0.06901 g copper(II) chloride (anhydrous) were added to this solution, forming a yellowish-dark green transparent solution. The mixture was heated for 2 hours under reflux to 62 to 65° C., with a blue precipitate being produced after about 5 minutes. The solution remained yellowish-dark green. The reaction mixture was left to cool and stirred for a further 48 hours at ambient temperature by means of a magnetic stirrer. A yellowish-dark green solution with precipitate was produced. The whole solution was filled into centrifugal glasses and centrifuged for 4 minutes at 4,000 revolutions/minute. A blue substance remained as sediment. The supernatant solution showed yellowy-green coloring. The supernatant solution was discarded. 5 ml of ethanol (pure) were added to the sediment, shaken and the solid was centrifuged off again for 4 minutes at 4,000 revolutions/minute. The supernatant solution showed yellowy-green coloring and was discarded. 5 ml of ethanol (pure) were again added to the sediment, shaken and the solid was centrifuged off for 4 minutes at 4,000 revolutions/minute. The yellow-green solution was discarded. Purification was repeated until the supernatant solution was colorless, with a total of approximately 25 ml ethanol (pure) usually being required for this purpose. The solid was dried in air and after drying a light blue, visibly homogenous product was obtained which could easily be efficiently and quite finely pulverized.

The solubility behavior of the copper-tegafur compound obtained substantially matched that of a copper(II) salt. Light blue coloring of the solution was obtained in water and the formation of a foam on shaking. The properties of the substance point to the fact that the compound Cu(Tf)₂ may have a salty character.

FIG. 4 shows the synthesis of Cu(Tf)₂ using a scheme.

### 3. Anti-Tumor Effectiveness of Cu(mel)₂

Using a murine renal cell carcinoma model (RENCA) of the Balb/c mouse a test of the effectiveness of mel-Cu-mel was carried out on the primary tumor, the metastases and the vessel density. For this the tumor cells were applied orthotopically into the left-hand peri-renal capsule of syngenic mice, the administered dose per day of treatment being: 5 mg/kg mouse and 7.5 mg/kg mouse, 5 animals per dose plus 5 animals for solvent control. The anti-tumor agent according to the invention Cu(mel)₂ was applied intraperitonial. The treatment model was as follows:

| | |
|---|---|
| Day 0 | application of the tumor cells |
| Day 1-9 | growth of the tumor |
| Day 10-18 | administration of the substance |
| Day 19 | killing of animals and findings. |

As a control 5 animals were used to which no active ingredient was administered and for comparison a group of 5 animals were administered melphalan alone.

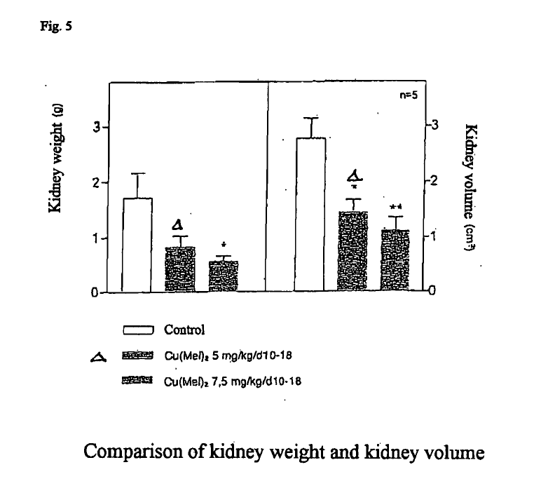

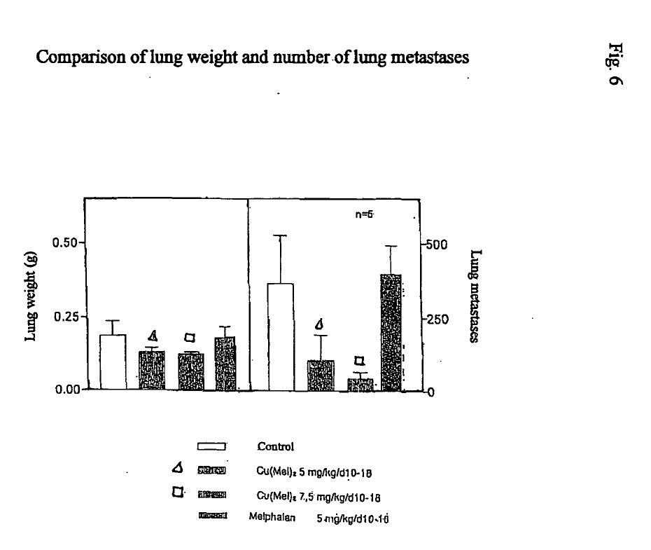

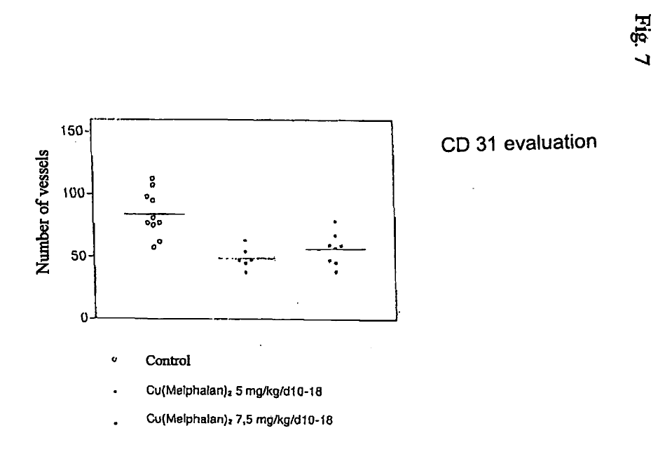

FIGS. 5 to 7 show the results of the investigation. It can be seen therefrom that, compared with the control, the compound Cu(mel)₂ according to the invention led to a significantly lower kidney weight and kidney volume (see FIG. 5), and this shows the effectiveness of the compounds according to the invention in treating malignant tumors. It may also be seen from FIG. 6 that the lung weight of mice treated with Cu(mel)₂, compared with the control and compared with mice to which melphalan alone was administered, is reduced. The advantageous effect of the compounds according to the invention on the metastases formation in the lungs is also particularly clear from the right-hand portion of FIG. 6 where the lung metastases formation of the mice treated according to the invention, the control mice and the mice treated with melphalan only is compared.

The mice treated according to the invention showed considerably lower lung metastases formation than the control mice and than the mice treated with melphalan only.

The test mice were also investigated with respect to vessel density, as is shown in FIG. 7. It may clearly be seen therefrom that the compounds according to the invention have an antiangiogenic effect.

### 4. In Vivo Tests on Anti-Tumoral and Antiangiogenic Effectiveness of Copper-Melphalan on an Orthotopic Renal Cell Carcinoma Model (RENCA) of a Mouse

The tests were carried out on a murine renal cell carcinoma model (RENCA) of the Balb/c mouse. The tumor cells were applied orthotopically into the left-hand renal capsule of syngenic mice. The effect of the treatment carried out on a primary tumor (in the implanted kidney), metastases (predominantly in the lungs, spleen and intestinal lymph nodes) and vessel density were investigated.

### Method:

After anesthesia by means of isoflurane narcosis (O₂— flow approx. 2.0 l with 0.5-1.5% isoflurane) a small incision was made in the mouse's left flank. The kidney was prepared and presented free from the peritoneum. A 27 G needle was introduced via the lower kidney pole through to below the renal capsule with visual inspection, and 4×10 tumor cells in 70 μl aliquots respectively applied once. The occurrence of a visible liquid-filled blister between kidney parenchyma and renal capsule and the discoloration of the kidneys were regarded as criteria for successful application. The kidney was subsequently reset and the incision into the abdominal wall closed by means of fascia stitches and cutaneous stitches with absorbable stitching material. Five test groups with ten animals respectively were formed. Group 1 negative control, group 2 positive control melphalan (Glaxo), group 3 and 4 MOC melphalan 2.5 mg/kg and 5 mg/kg. In the case of group 5 an antiangiogenically effective substance was applied as the positive control. The start of treatment was fixed for day 1 post op. for Cu(melphalan)₂. Treatment was carried out daily up to day seven (see treatment model). The test was ended after 19 days since, owing to their size, the control tumors required termination for ethical reasons.

### Treatment Model:

| | | Treatment | | Number of |
|---|---|---|---|---|
| Test groups | Dose | days | Application | animals |
| Control | — | — | | 10 |
| Positive control | 5 mg/kg/d | Day 1-7 | i.p. | 10 |
| melphalan | (16.4 μmol/kg) | | | |
| Cu(melphalan)₂ | 2.5 mg/kg | Day 1-7 | i.p. | 10 |
| | (4.4 μmol/kg) | | | |
| Cu(melphalan)₂ | 5 mg/kg/d | Day 1-7 | i.p. | 10 |
| | (8.8 μmol/kg) | | | |
| Antiangiogenic | 50 mg/kg/d | Day 1-19 | p.o. | 10 |
| substance | | | | |

The implantation day was counted as day 0.
Duration of the test 19 days.
Number of animals at beginning of test 50, no animals died during the op. 3 animals from the control group were found to have perished on the 14^{th} or 15^{th} day.
The chosen dosage of test substance was guided by an earlier preliminary test for determining dosage effectiveness.

### Primary Tumor

Data Sheet Kidney Volume (cm³)

Test groups

| | Positive control | | |
|---|---|---|---|
| | Melphalan | Cu(melphalan)₂ | Cu(melphalan)₂ |
| Control | 16.4 μmol/kg | 4.4 μmol/kg | 8.8 μmol/kg |
| 2.52 | 2.00 | 1.53 | 1.30 |
| — | 0.37 | 1.71 | 1.12 |
| 3.10 | 0.77 | 1.71 | 0.6 |
| 2.34 | 1.26 | 1.80 | 0.64 |
| 3.75 | 1.60 | 0.60 | 1.26 |
| 4.29 | 2.69 | 1.54 | 1.24 |
| — | 0.58 | 2.2 | 0.44 |
| 3.45 | 1.3 | 1.2 | 1.5 |
| — | 0.28 | 0.96 | 1.2 |
| 3.55 | 1.76 | 2.13 | 1.33 |

### Data Sheet Kidney Weight (gr)

Test groups

| | Positive control | | |
|---|---|---|---|
| | Melphalan | Cu(melphalan)₂ | Cu(melphalan)₂ |
| Control | 16.4 μmol/kg | 4.4 μmol/kg | 8.8 μmol/kg |
| 1.72 | 1.23 | 1.02 | 0.74 |
| — | 0.12 | 1.28 | 0.79 |
| 1.50 | 0.34 | 1.06 | 0.18 |
| 1.79 | 0.40 | 1.15 | 0.29 |
| 3.26 | 0.65 | 0.29 | 0.36 |
| 3.17 | 1.70 | 0.78 | 0.79 |
| — | 0.38 | 2.00 | 0.19 |
| 3.14 | 0.44 | 0.81 | 1.06 |
| — | 0.13 | 0.38 | 0.59 |
| 2.46 | 1.02 | 1.58 | 0.85 |

### Lung Metastases

### Data Sheet: Number of Lung Metastases

Test groups

| | Positive control | | |
|---|---|---|---|
| | Melphalan | Cu(melphalan)₂ | Cu(melphalan)₂ |
| Control | 16.4 μmol/kg | 4.4 μmol/kg | 8.8 μmol/kg |
| 20 | 5 | >750 | 16 |
| — | 640 | 49 | 610 |
| >750 | >750 | 29 | 92 |
| >750 | 328 | 3 | 4 |
| 4 | 28 | 570 | 7 |
| >750 | 7 | 378 | 11 |
| — | 273 | >750 | 4 |
| >750 | 1 | >750 | 162 |
| — | >750 | >750 | 0 |
| 278 | 28 | 9 | 680 |

Data Sheet Lung Weight (gr)

Test groups

| | Positive control | | |
|---|---|---|---|
| | Melphalan | Cu(melphalan)₂ | Cu(melphalan)₂ |
| Control | 16.4 μmol/kg | 4.4 μmol/kg | 8.8 μmol/kg |
| 0.14 | 0.10 | 0.14 | 0.31 |
| — | 0.25 | 0.24 | 0.14 |
| 1.44 | 0.48 | 0.11 | 0.22 |
| 0.46 | 0.22 | 0.13 | 0.18 |
| 0.11 | 0.14 | 0.13 | 0.13 |
| 0.77 | 0.12 | 0.20 | 0.14 |
| — | 0.20 | 0.31 | 0.16 |
| 0.96 | 0.13 | 0.49 | 0.15 |
| — | 0.76 | 1.07 | 0.11 |
| 0.18 | 0.12 | 0.11 | 0.21 |

### Lymph Node Metastases

### Data Sheet: Number of Intestinal Lymph Node Metastases

Test groups

| | Positive control | | |
|---|---|---|---|
| | Melphalan | Cu(melphalan)₂ | Cu(melphalan)₂ |
| Control | 16.4 μmol/kg | 4.4 μmol/kg | 8.8 μmol/kg |
| 5 | 0 | 0 | 4 |
| — | 0 | 0 | 0 |
| — | 0 | 0 | 0 |
| 8 | 0 | 6 | 2 |
| 120 | 5 | 0 | 0 |
| 45 | 0 | 1 | 0 |
| — | 9 | 2 | 0 |
| 52 | 0 | 3 | 0 |
| — | 2 | 4 | 0 |
| 12 | 8 | 0 | 0 |

Vessel Density (Anti-CD31 AB)

### Method:

After selecting the animals the tissues to be examined were quickly transferred into liquid nitrogen and then frozen at −80° C. 5-10 μm thick sections were prepared from the tissue parts. The preparations were immunohistochemically dyed with an anti-CD31 antibody (Pecam-1) and the number of vessels in the control and treatment groups counted. Two sections of each tumor with three areas respectively were used for counting the vessels.

### Data Sheet: Vessel Density (Mean of Three Counted Areas Per Section)

Test groups

| | Positive control | | |
|---|---|---|---|
| | Melphalan | Cu(melphalan)₂ | Cu(melphalan)₂ |
| Control | 16.4 μmol/kg | 4.4 μmol/kg | 8.8 μmol/kg |
| 77 | 57 | 54 | 54 |
| 75 | 80 | 61 | 63 |
| — | — | 33 | 86 |
| — | — | 46 | 91 |
| 62 | 72 | 62 | — |
| 60 | 56 | 46 | — |
| 70 | 56 | 37 | 39 |
| 72 | 49 | 68 | 48 |
| 57 | 74 | 57 | 55 |
| 64 | 65 | 63 | 50 |
| 54 | 62 | 108 | 90 |
| 50 | 114 | 181 | 122 |
| — | 32 | 124 | 31 |
| — | 52 | 132 | 44 |
| — | 25 | 123 | 67 |
| — | 28 | 122 | 57 |
| — | 57 | 82 | — |
| — | 44 | 88 | — |
| 28 | 107 | 68 | 63 |
| 35 | 127 | 113 | 45 |

No toxicities were observed during the test and there were no significant reductions in the body weight either as an indication of side effects. No particular pathological findings could be found on dissection.

Cu(melphalan)₂ has a significant anti-tumor effect in a dose of 2.5 mg/kg and 5 mg/kg, wherein with an approximately semi-equimolar dose it demonstrates better effectiveness than melphalan alone. Effectiveness comparable with melphalan was achieved with an approximately quarter-equimolar dose.

When evaluating the formed lung metastases, it was possible to prove a significant anti-metastatic effect of Cu(melphalan)₂ with a dose of 5 mg/kg in the test.

When evaluating the formed intestinal lymph node metastases, it was possible to prove a significant anti-metastatic effect of Cu(melphalan)₂ with a dose of 2.5 mg/kg and 5 mg/kg.

Using the selected treatment model it was not possible to observe any antiangiogenic effectiveness with the two doses. This finding correlates with the results of other cytostatically effective substances in this model.

### Example 5

### Study Protocol

The study report matches that of Example 4, differing only in terms of treatment intervals and dose groups. Four test groups with ten animals respectively were formed. Group 1 negative control, group 2 positive control melphalan (Glaxo), groups 3 and 4 Cu(melphalan)₂ 5 mg/kg and 7.5 mg/kg. The start of treatment was fixed for day 10 post op. for all groups. The intention was for daily treatment from day 10 until day 17 (see treatment model). Owing to toxicities the treatment period was reduced to day 10-14. After 18 days the test was ended since, owing to their size, the control tumors required termination for ethical reasons.

### Treatment Model:

| | | Treatment | | |
|---|---|---|---|---|
| Test groups | Dose | days | Application | Number |
| Control | — | — | | 10 |
| Positive control | 5 mg/kg/d | Day 10-17 | i.p. | 10 |
| Melphalan | (16.4 μmol/kg) | | | |
| Cu(melphalan)₂ | 5 mg/kg | Day 10-17 | i.p. | 10 |
| | (8.8 μmol/kg) | | | |
| Cu(melphalan)₂ | 7.5 mg/kg/d | Day 10-17 | i.p | 10 |
| | (13.2 μmol/kg) | | | |

The implantation day was counted as day 0.
Duration of the test 18 days.
Number of animals at beginning of test 40; no animals died during the op.

### Results:

### Primary Tumor

Data Sheet Kidney Volume (cm³)

Test groups

| | Positive control | | |
|---|---|---|---|
| | Melphalan | Cu(melphalan)₂ | Cu(melphalan)₂ |
| Control | 16.4 μmol/kg | 8.8 μmol/kg | 13.2 μmol/kg |
| 4.25 | 2.20 | 2.90 | 0.91 |
| — | 3.91 | — | 3.06 |
| 4.41 | 3.63 | — | 1.22 |
| 3.45 | 2.80 | 1.96 | 3.33 |
| 6.30 | 3.23 | 2.76 | 1.68 |
| 2.55 | 3.94 | 3.12 | 1.96 |
| 5.03 | 2.59 | 2.60 | 3.08 |
| 3.74 | 2.08 | — | 0.60 |
| 2.95 | 4.25 | 2.63 | 2.60 |
| 5.87 | 2.84 | 2.08 | 1.98 |

### Data Sheet Kidney Weight (gr)

Test groups

| | Positive control | | |
|---|---|---|---|
| | Melphalan | Cu(melphalan)₂ | Cu(melphalan)₂ |
| Control | 16.4 μmol/kg | 8.8 μmol/kg | 13.2 μmol/kg |
| 2.79 | 1.63 | 1.78 | 0.27 |
| — | 2.11 | — | 1.92 |
| 3.01 | 1.90 | — | 0.46 |
| 2.51 | 1.45 | 0.75 | 2.50 |
| 4.30 | 1.13 | 1.56 | 0.74 |
| 1.43 | 2.77 | 1.70 | 1.37 |
| 3.41 | 1.38 | 1.78 | 2.39 |
| 2.48 | 0.69 | — | 0.26 |
| 1.95 | 2.91 | 1.88 | 1.90 |
| 3.92 | 1.90 | 1.23 | 1.05 |

### Lung Metastases

### Data Sheet: Number of Lung Metastases (Late Treatment Day 10-14)

Test groups

| | Positive control | | |
|---|---|---|---|
| | Melphalan | Cu(melphalan)₂ | Cu(melphalan)₂ |
| Control | 16.4 μmol/kg | 8.8 μmol/kg | 13. 2 μmol/kg |
| 92 | 71 | 431 | 77 |
| — | 750 | — | 750 |
| 750 | 500 | — | 300 |
| 750 | 46 | 750 | 4 |
| 750 | 750 | 0 | 10 |
| 750 | 304 | 28 | 0 |
| 128 | 614 | 0 | 3 |
| 182 | 317 | — | 0 |
| 0 | 5 | 21 | 0 |
| 87 | 750 | 345 | 0 |

### Data Sheet Lung Weight (gr)

Test groups

| | Positive control | | |
|---|---|---|---|
| | Melphalan | Cu(melphalan)₂ | Cu(melphalan)₂ |
| Control | 16.4 μmol/kg | 8.8 μmol/kg | 13.2 μmol/kg |
| 0.13 | 0.11 | 0.13 | 0.13 |
| — | 0.38 | — | — |
| 0.61 | — | — | — |
| 0.78 | 0.10 | 0.21 | — |
| 0.31 | 0.31 | 0.13 | — |
| 0.40 | 0.14 | 0.11 | 0.11 |
| 0.14 | 0.15 | 0.14 | 0.13 |
| 0.13 | 0.13 | — | 0.10 |
| 1.10 | 0.13 | 0.11 | — |
| 0.11 | 0.28 | 0.15 | — |

### Lymph Node Metastases

### Data Sheet: Number of Intestinal Lymph Node Metastases

Test groups

| | Positive control | | |
|---|---|---|---|
| | Melphalan | Cu(melphalan)₂ | Cu(melphalan)₂ |
| Control | 16.4 μmol/kg | 8.8 μmol/kg | 13.2 μmol/kg |
| 0 | 17 | 64 | 0 |
| — | 22 | — | 0 |
| 50 | — | — | 0 |
| 42 | 32 | 51 | 0 |
| 32 | 84 | 75 | 0 |
| 28 | 91 | 20 | 0 |
| 8 | 0 | 40 | 0 |
| 109 | 0 | — | 0 |
| 120 | 0 | 36 | 0 |
| 22 | 49 | 12 | 0 |

### Data Sheet: Vessel Density, Later Start of Treatment Day 10-14

Test groups

| | Positive control | | |
|---|---|---|---|
| | Melphalan | Cu(melphalan)₂ | Cu(melphalan)₂ |
| Control | 16.4 μmol/kg | 8.8 μmol/kg | 13.2 μmol/kg |
| 52 | 64 | 25 | 58 |
| 62 | 62 | 56 | 43 |
| 47 | 40 | 35 | 48 |
| 56 | 40 | 40 | 65 |
| 65 | 34 | 31 | 53 |
| 65 | 36 | 44 | 35 |
| 27 | 28 | 44 | 35 |
| 60 | 36 | 38 | 52 |
| 59 | 42 | 34 | — |
| 56 | 41 | 56 | — |
| 69 | 68 | 46 | — |
| 42 | 35 | 26 | — |
| 62 | 47 | 51 | — |
| 57 | 32 | 72 | — |
| 58 | 37 | — | — |
| 52 | — | — | — |

The treatment was terminated on day 14 for reasons of tolerability. Cu(melphalan)₂ has a significant anti-tumoral effect against the primary tumor in a dose of both 8.8 μmol/kg and 13.2 μmol/kg. A significant anti-metastatic effect of a dose of 13.2 μmol/kg was found with respect to lung metastases and lymph node metastases. Cu(melphalan)₂ is also significantly antiangiogenic in a dose of 8.8 μmol/kg.

### Example 6

### The Effectiveness of Cu(Melphalan)₂ Compared with Melphalan in NOD/SCID Mice Injected with OVCAR-3 Tumor Cells

2×10⁶ OVCAR-3/P19 tumor cells were s.c. injected into female NOD/SCID mice. The mice were treated as follows.

| | | Subst. | | Dose |
|---|---|---|---|---|
| Gr. | Mice | i.p. | Treatment days | [mg/kg] |
| 1 | 8 | PBS | 20, 21, 25, 29, 33, 37 | / |
| 2 | 8 | Melphalan | 21, 23, 25, 27, 29, 31, 33, 35, 37 | 5 |
| 3 | 8 | Copper- | 21, 23, 25, 27, 29, 31, 33, 35, 37 | 7.5 |
| | | melphalan | | |

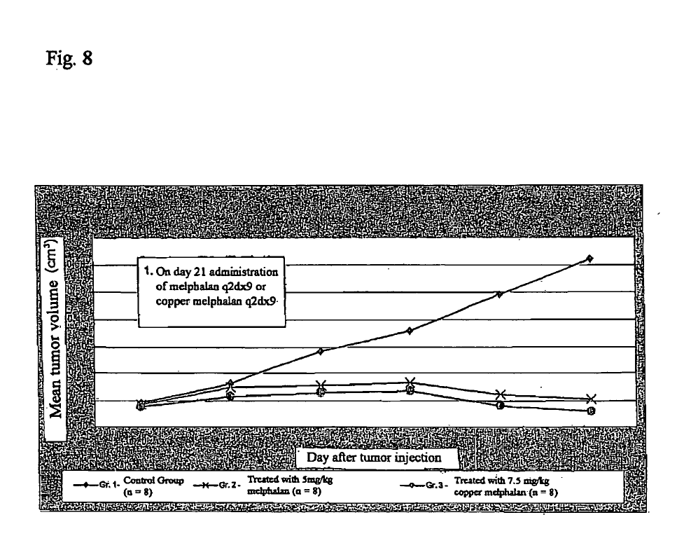

The treatment began with a mean tumor volume of 80 mm³. Stable and constant tumor growth was observed in the control group. FIG. 8 shows the mean tumor volume during the course of treatment.

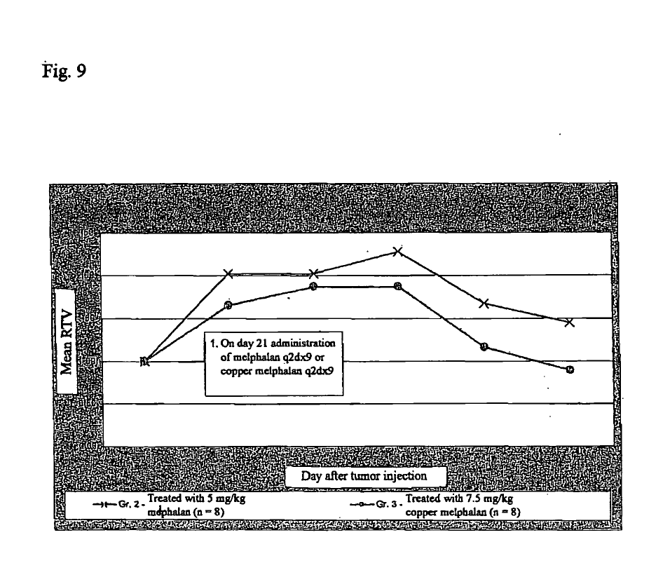

To level possible differences in tumor volume at the start of treatment in the individual groups and to make evident differences owing to the low absolute volumes in the treatment groups, the relative tumor volumes (RTV) have been analyzed. The relative tumor volume results from the ratio of the volume on day x and the tumor volume at the start of treatment in the same test animal. FIG. 9 shows the RTVs from the start of treatment for the groups treated with chemotherapeutics. This clearly shows that copper-melphalan is more effective than melphalan. Only 61.3% of group 2 or 8.2% of the control achieved the mean RTV on day 37 of group 3 (copper-melphalan). The tumor growth was stopped in group 2 after just 7 days. After 3 days of constant tumor volume a tumor regression began from day 30 (10^{th} day of treatment) in the test animals treated with copper-melphalan.

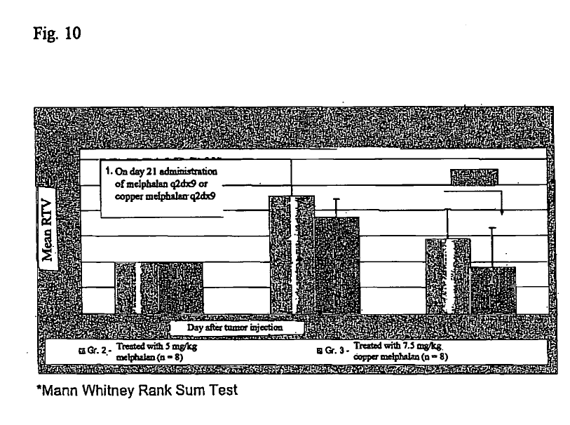

FIG. 10 shows a statistical analysis of the two dose groups. On day 37 both treatment groups (groups 2 and 3) demonstrated a statistical significance (p<0.01, student's t-test) in the case of tumor volume and relative tumor volume compared with the control group. In addition a statistically significant higher degree of effectiveness of copper-melphalan compared with melphalan could be confirmed at the end of treatment (day 37). The p values for the mean tumor volume or the mean RTV on day 37, tested using a Mann-Whitney rank sum test, were p=0.021 and p=0.01.

As the following table shows, it was also found that, in contrast to treatment with copper-melphalan, the tumor volume in mice which were treated with melphalan only was greater at the end of the study than the tumor volume at the start of treatment. On day 37, 6 of 8 mice (75%) had a RTV>1. Overall the test animals of group 2 had between 20% tumor regression and 98% growth at the end of treatment. In group 3 one test animal had an RTV of 2.78, all others were in a range from 0.43 to 0.78. A tumor regression between 22% and 57% could thus be observed in 88% of the mice in group 3 on day 37. Both the frequency of tumor regression and the strength of regression was much higher in the group treated with copper-melphalan.

### Group 1

| | | Day 20 | | Day 30 | | Day 37 | |
|---|---|---|---|---|---|---|---|
| | | Vol | RTV | Vol | RTV | Vol | RTV |
| Gr. 1 | 1 | 0.15 | 1.00 | 0.55 | 3.67 | 0.79 | 5.27 |
| Control | 2 | 0.07 | 1.00 | 0.27 | 3.86 | 0.85 | 12.14 |
| (n = 8) | 3 | 0.11 | 1.00 | 0.56 | 5.09 | 0.82 | 7.47 |
| | 4 | 0.26 | 1.00 | 0.68 | 2.62 | 0.92 | 3.54 |
| | 5 | 0.01 | 1.00 | 0.17 | 17.00 | 0.28 | 28.40 |
| | 6 | 0.02 | 1.00 | 0.15 | 7.50 | 0.28 | 14.05 |
| | 7 | 0.05 | 1.00 | 0.21 | 4.20 | 0.34 | 6.88 |
| | 8 | 0.08 | 1.00 | 0.28 | 3.50 | 0.69 | 8.66 |

| | | | | | | |
|---|---|---|---|---|---|---|
| Mean | 0.09 | 1.00 | 0.36 | 5.93 | 0.62 | 10.80 |
| Standard deviation | 0.08 | 0.00 | 0.21 | 4.71 | 0.27 | 7.90 |
| Median | 0.08 | 1.00 | 0.28 | 4.03 | 0.74 | 8.07 |

### Group 2

| | | Day 20 | | Day 30 | | Day 37 | |
|---|---|---|---|---|---|---|---|
| | | Vol | RTV | Vol | RTV | Vol | RTV |
| Gr. 2 | 1 | 0.03 | 1.00 | 0.09 | 3.00 | 0.07 | 2.37 |
| treated with | 2 | 0.10 | 1.00 | 0.14 | 1.40 | 0.08 | 0.81 |
| 5 mg/kg | 3 | 0.20 | 1.00 | 0.35 | 1.75 | 0.21 | 1.05 |
| melphalan | 4 | 0.05 | 1.00 | 0.16 | 3.20 | 0.08 | 1.66 |
| (n = 8) | 5 | 0.12 | 1.00 | 0.17 | 1.42 | 0.10 | 0.82 |
| | 6 | 0.10 | 1.00 | 0.22 | 2.20 | 0.17 | 1.71 |
| | 7 | 0.04 | 1.00 | 0.10 | 2.50 | 0.05 | 1.18 |
| | 8 | 0.04 | 1.00 | 0.11 | 2.75 | 0.08 | 1.98 |

| | | | | | | |
|---|---|---|---|---|---|---|
| Mean | 0.09 | 1.00 | 0.17 | 2.28 | 0.11 | 1.45 |
| Standard deviation | 0.06 | 0.00 | 0.09 | 0.70 | 0.06 | 0.57 |
| Median | 0.08 | 1.00 | 0.15 | 2.35 | 0.08 | 1.42 |

### Group 3

| | | Day 20 | | Day 30 | | Day 37 | |
|---|---|---|---|---|---|---|---|
| | | Vol | RTV | Vol | RTV | Vol | RTV |
| Gr.3 | 1 | 0.11 | 1.00 | 0.15 | 1.36 | 0.05 | 0.43 |
| treated | 2 | 0.05 | 1.00 | 0.11 | 2.20 | 0.14 | 2.78 |
| with 7.5 | 3 | 0.04 | 1.00 | 0.07 | 1.75 | 0.03 | 0.78 |
| mg/kg | 4 | 0.10 | 1.00 | 0.19 | 1.90 | 0.07 | 0.67 |
| copper- | 5 | 0.02 | 1.00 | 0.05 | 2.50 | 0.01 | 0.70 |
| melphalan | 6 | 0.09 | 1.00 | 0.16 | 1.78 | 0.05 | 0.60 |
| (n = 8) | 7 | 0.12 | 1.00 | 0.22 | 1.83 | 0.06 | 0.51 |
| | 8 | 0.08 | 1.00 | 0.13 | 1.63 | 0.05 | 0.63 |

| | | | | | | |
|---|---|---|---|---|---|---|
| Mean | 0.08 | 1.00 | 0.14 | 1.87 | 0.06 | 0.89 |
| In % of group 1 | 81.3% | 100.0% | 37.6% | 31.5% | 9.3% | 8.2% |
| In % of group 2 | 89.7% | 100.0% | 80.6% | 82.1% | 55.1% | 61.3% |
| Standard deviation | 0.04 | 0.00 | 0.06 | 0.35 | 0.4 | 0.77 |
| Median | 0.09 | 1.00 | 0.14 | 1.81 | 0.05 | 0.65 |
| In % of group 1 | 113.3% | 100.0% | 50.9% | 44.8% | 7.0% | 8.0% |
| In % of group 2 | 113.3% | 100.0% | 93.3% | 76.8% | 63.4% | 45.7% |

1. A use of a compound of formula (I)
Cu(L)₂.(H₂O)ₓ (I)
wherein
L is independently selected from melphalan (4-[bis(2-chloroethyl)amino]-L-phenylalanine) and tegafur (5-fluoro-1-(tetrahydro-2-furyl)-uracil) for each x; and
x=0, 1 or 2,
for preparing a medicament for preventing tumors, treating tumors, or both.

2. The use of the compound of formula (I) as claimed in claim 1, wherein both L are L melphalan or both L are L tegafur.

3. The use of the compound of formula (I) as claimed in claim 1, wherein both L are L melphalan.

4. The use of the compound of formula (I) as claimed in claim 1, wherein x=2.

5. The use of the compound of formula (I) as claimed in claim 1 for treating at least one of the colon cancer, brain tumors, eye tumors, pancreatic carcinomas, bladder carcinomas, lung tumors, breast cancer, ovarian tumors, cancer of the uterus, bone tumors, gall bladder and bile duct carcinomas, head-neck tumors, skin cancer, testicular cancer, kidney tumors, germ cell tumors, liver cancer, leukemia, malignant lymphoma, never tumors, neuroblastomas, prostate cancer, soft tissue tumors, esophageal cancer and carcinomas in the case of unknown primary tumors.

6. The use of the compound of formula (I) as claimed in claim 5 for treating at least one of kidney and lung tumors.

7. The use of the compound of formula (I) as claimed in claim 1, wherein the compound of formula (I) is in a form suitable for topical, parenteral, intravenous, intramuscular, subcutaneous or transdermal administration.

8. The use of the compound of formula (I) as claimed in claim 1, wherein the compound of formula (I) is administered together with pharmaceutically acceptable adjuvants.
